(19) United States
(12) Patent Application Publication
(10) Pub. No.: US 2019/0133769 A1
(43) Pub. Date: May 9, 2019

(54) TRUSS IMPLANT WITH ROUNDED CORNERS

(71) Applicants: Kevin D. Tetsworth, Toowong (AU); Vaida Glatt, San Antonio, TX (US)

(72) Inventors: Kevin D. Tetsworth, Toowong (AU); Vaida Glatt, San Antonio, TX (US)

(21) Appl. No.: 15/804,008

(22) Filed: Nov. 6, 2017

(51) Int. Cl. A61F 2/28; A61F 2/30; A61F 2/46

(52) U.S. Cl. CPC A61F 2/2846; A61F 2/30907; A61F 2/32; A61F 2002/30878; A61F 2/4611

(57) ABSTRACT

An implant includes an implant body including a contact face configured to be disposed at or near a bony structure, and a truss structure coupled to the contact face. The truss structure includes a plurality of struts which extend from the contact face and which form a plurality of polygons that have rounded corners.

### FIELD OF THE INVENTION

The present invention generally relates to bone implants, and particularly to a bone implant that has a truss made of struts with rounded corners.

### BACKGROUND OF THE INVENTION

Implants for supporting bony structures in the body are known. Implants may be formed as a three-dimensional structure for use as a bone mass replacement or supporting bony tissue. Implants may be constructed from interconnecting metallic segments or filaments that are joined to form regular repeating geometric shapes to provide strength throughout the structure. For example, the geometric shapes may include a dodecahedron or a modified dodecahedron based on pentagons. The resulting structure may have a general rectangular block configuration, a cylindrical configuration or other shapes.

The present inventors have identified a possible problem that has gone unnoticed in the prior art. The segments, filaments, trusses and the like of the prior art structures all have intersecting nodes with sharp corners or angles. This can be problematic because sharp corners may inhibit cell growth. For example, US Patent Application 20010037150 uses sharp corners to inhibit cell growth, thereby to prevent epithelial cell growth that may lead to posterior capsule opacification in the eye.

### SUMMARY OF THE INVENTION

The present invention seeks to provide an improved implant with a truss structure that has rounded corners, as is described more in detail hereinbelow. In contrast to the prior art, the truss structure with rounded corners encourages, rather than discourages, cell growth at the nodes of the truss structure and throughout the truss structure. This can provide significant improvements over the prior art truss structures in terms of bone growth in the implant between the trusses, thereby enhancing and accelerating the healing process and improving the ability of the implant to support bony structures in the body.

There is provided in accordance with a non-limiting embodiment of the invention an implant including an implant body including a contact face configured to be disposed at or near a bony structure, and a truss structure coupled to the contact face, the truss structure including a plurality of struts which extend from the contact face and which form a plurality of polygons that have rounded corners.

The polygons may include Voronoi polygons. The struts may have a rounded cross-sectional shape, such as but not limited to, circular, elliptic or irregular.

In accordance with a non-limiting embodiment of the invention, not all of the polygons have identical numbers of sides. Alternatively, they may all have the same number of sides.

In accordance with a non-limiting embodiment of the invention a method includes disposing an implant body including a contact face at or near a bony structure, the contact face having a truss structure coupled thereto, the truss structure including a plurality of struts which extend from the contact face and which form a plurality of polygons that have rounded corners.

### BRIEF DESCRIPTION OF THE DRAWINGS

The present invention will be understood and appreciated more fully from the following detailed description taken in conjunction with the drawings in which:

FIG. 1 is a simplified illustration of an implant having a truss structure, constructed and operative in accordance with a non-limiting embodiment of the present invention; and

FIGS. 2 and 3 are simplified illustrations of truss structures made with Voronoi polygons with rounded corners, constructed and operative in accordance with a non-limiting embodiment of the present invention; and

FIGS. 4, 5 and 6 are some cross-sectional shapes of struts of the truss structure, in accordance with different non-limiting embodiments of the present invention.

### DETAILED DESCRIPTION OF EMBODIMENTS

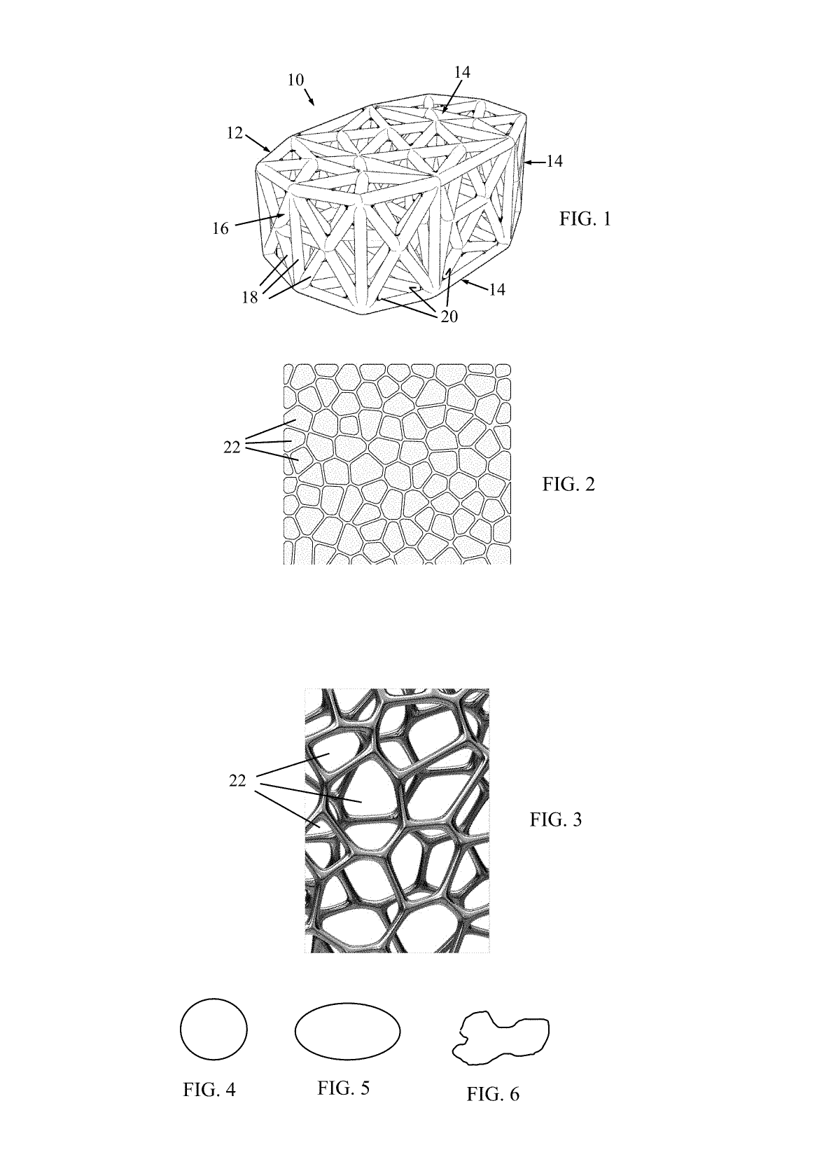

Reference is now made to FIG. 1, which illustrates an implant 10, in accordance with a non-limiting embodiment of the present invention.

Implant 10 includes an implant body 12 which has one or more contact faces 14 configured to be disposed at or near a bony structure. A truss structure 16 is coupled to the contact face(s) 14. Truss structure 16 includes a plurality of struts 18 which extend from the contact face(s) 14 and which form a plurality of polygons that have rounded corners 20. None of the nodes at the intersections of the struts have sharp corners, thereby encouraging cell growth at the nodes of the truss structure 16 and throughout the truss structure 16.

The polygons in FIG. 1 are regular polygons, such as triangles, rectangles, pentgone, hexagons, etc.

Reference is now made to FIGS. 2 and 3, which illustrate truss structures made with Voronoi polygons 22 with rounded corners.

By definition, a Voronoi polygon is the partitioning of a plane into convex polygons such that each polygon contains exactly one generating point and every point in a given polygon is closer to its generating point than to any other. Other terms which are used interchangeably herein with Voronoi polygon are Voronoi diagram or pattern, or Dirichlet tessellation. Voronoi patterns can be seen in nature such as the spots on a giraffe's body or a dragonfly's wings.

Without limitation, the construction of the implant with Voronoi polygons may be carried out by modifying the methods of U.S. Pat. No. 6,133,921, the disclosure of which is incorporated herein by reference. In the method of U.S. Pat. No. 6,133,921, starting with a solid model of the desired shape of the three-dimensional implant (e.g., cylinder, dodecahedron, oblong shape, etc.), an accurate skeleton is produced by minimizing internal and edge errors of an initial approximate skeleton of the implant. To produce the initial approximate skeleton, the boundary of the implant is densely sampled to obtain a set of surface data points. Delaunay triangulation is performed on the surface data points and exterior and spurious tetrahedra are removed, leaving substantially interior tetrahedra. Circumspheres are constructed that enclose the tetrahedra, and the centers of the circumspheres are connected based on tetrahedra adjacency, to form a Voronoi diagram. Closed Voronoi cells are identified comprising the initial approximate skeleton. The position of skeleton interior vertices are then adjusted to minimize their error, producing a refined polygonal approximation of the skeleton interior geometry. Positions of the skeleton edge vertices are adjusted to minimize the edge error, yielding an accurate polygonal approximation of the skeleton.

In contrast to U.S. Pat. No. 6,133,921, the Voronoi polygons are modified to have rounded corners. This may be carried out with commercially available software used to round corners of Voronoi polygons, such as:

http://www.craftsmanspace.com/free-patterns/mathematical-2d-patterns.html

http://www.craftsmanspace.com/free-patterns/voronoi-2d-patterns.html

The struts may have a rounded cross-sectional shape, such as but not limited to, circular (FIG. 4), elliptic (FIG. 5) or irregular (FIG. 6).

In accordance with a non-limiting embodiment of the invention, not all of the polygons have identical numbers of sides. Alternatively, they may all have the same number of sides.

The implants of the invention may include, without limitation, spinal implants, corpectomy devices, hip replacements, knee replacements, long bone reconstruction scaffolding, cranio-maxifacial implants and many more.

What is claimed is:

1. An implant comprising:
an implant body comprising a contact face configured to be disposed at or near a bony structure; and
a truss structure coupled to said contact face, said truss structure comprising a plurality of struts which extend from said contact face and which form a plurality of polygons that have rounded corners.

2. The implant according to claim 1, wherein said polygons comprise Voronoi polygons.

3. The implant according to claim 1, wherein said struts have a rounded cross-sectional shape.

4. The implant according to claim 3, wherein said cross-sectional shape is circular.

5. The implant according to claim 3, wherein said cross-sectional shape is elliptic.

6. The implant according to claim 1, wherein not all of said polygons have identical numbers of sides.

7. A method comprising:
disposing an implant body comprising a contact face at or near a bony structure, said contact face having a truss structure coupled thereto, said truss structure comprising a plurality of struts which extend from said contact face and which form a plurality of polygons that have rounded corners.
